(19) United States
(12) Patent Application Publication
(10) Pub. No.: US 2003/0161876 A1
(43) Pub. Date: Aug. 28, 2003

(54) Granulation process and starch granulate

(76) Inventors: Henri Hansson, Helsingborg (SE); Elisabeth Lindner-Olsson, Hagersten (SE)

Correspondence Address: Benton S. Duffett, Jr., BURNS, DOANE, SWECKER & MATHIS, L.L.P., P.O. Box 1404, Alexandria, VA, 22313-1404, US

(21) Appl. No.: 10/108,561

(22) Filed: Mar. 29, 2002

(30) Foreign Application Priority Data
Feb. 25, 2002 (SE) 0200539-5

(51) Int. Cl. A61K 9/20; A61K 9/16; A61K 9/50; C08B 30/00

(52) U.S. Cl. 424/465; 514/60; 127/65

(57) ABSTRACT

A process for the manufacture of a corn starch granulate possessing resistance to enzymatic degradation upon oral administration, comprising the steps:

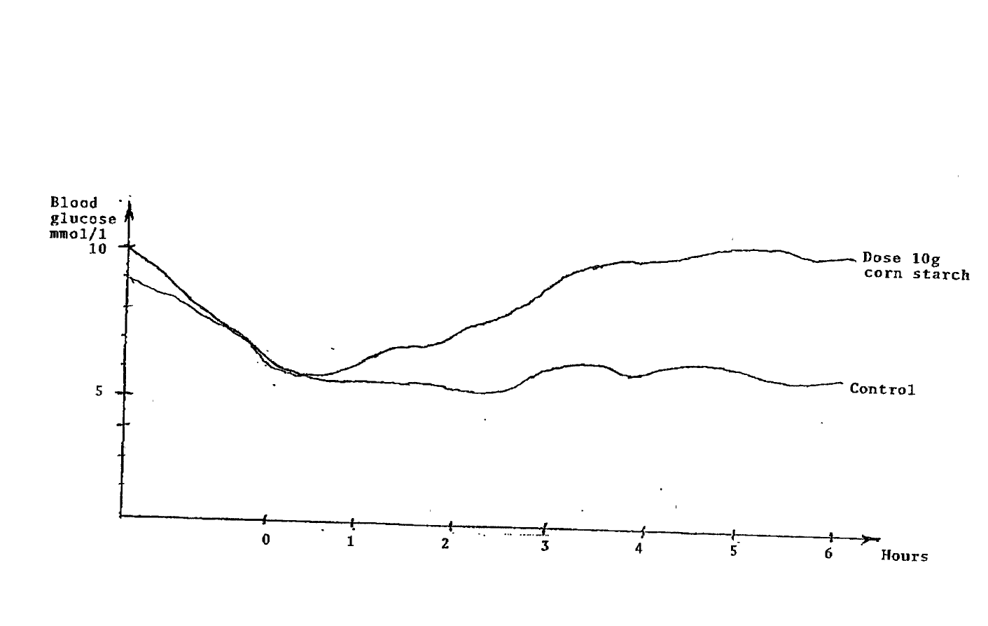

a) granulating native corn starch by cautious mixing of a granulation fluid comprising a binder ethanol or water as a solvent, corn starch and a sweetener;

b) subjecting the granulated material resulting from step a) to wet sieving;

c) drying the granulate obtained in step b) at a temperature less than about 55° C. to avoid gelatinization of the corn starch; and

d) sizing the dried granulate from step c) by dry sieving; and a

corn starch granulate comprising primary corn starch granules as they appear in untreated native corn starch, said primary granules being agglomerated, without degradation thereof, into larger secondary granules to form a granulate using a binder selected from pre-gelatinized corn starch, gum arabicum, guar gum, potassium alginate, carageenan, methyl cellulose, and ethyl cellulose.

The present invention relates to a process for the manufacture of corn starch granulates possessing resistance to enzymatic degradation upon oral administration. The invention also covers corn starch granulates having such resistance to enzymatic degradation.

### BACKGROUND OF THE INVENTION

Near normalization of blood glucose levels in diabetes is associated with around 50% less risk of nephropathy and retinopathy. However, the aim of normalizing the blood glucose levels is impeded by the risk of inducing hypoglycemia—the Achilles heel of diabetes management. Severe hypoglycemia is increased by 4-5 fold in patients with near-normal blood glucose levels by such normalization. A majority of these episodes occur during sleep. Nocturnal hypoglycemia represents a particularly threatening condition to the patient. At daytime, it is possible for the patient himself, or people in close proximity, to recognize and treat the early autonomic warning symptoms. At nighttime, however, when the patient is asleep the autonomic symptoms may not be enough to awaken the patient. Therefore an initially mild hypoglycemic episode may easily progress into a severe episode at a time when external assistance may not be available to the patient.

The causes of hypoglycemia may be recapitulated in a few key determinants. Available basal insulin formulations do not provide a constant and reliable insulin supply. Neither is food consumed in a standardized and consistent manner. Moreover, the nocturnal insulin effect reaches a peak around 3-5 AM, a time when the dietary glucose is absorbed and the risk of hypoglycemia is at its greatest. Lowering the insulin dose is not a practical alternative because of the loss of blood glucose control on the following day. In contrast, an oral therapy that provides a timed nocturnal glucose delivery may balance an excessive insulin effect without distorting blood glucose levels the following day.

In exploring the options to optimize the nocturnal glucose delivery different test-snacks have been tested. Normal snacks, such as bread or milk, produce a peak glucose delivery around 1-1½ hours after ingestion. Such a snack provokes early hyperglycemia without protecting against hypoglycemia after mid-night. A solution appears to be found in the use of particular starches characterized by a low rate of glucose delivery. Native corn starch has a peak glucose delivery at ˜4 hours. Corn starch is used to avert nocturnal hypoglycemia in young children with glycogen storage disease. Corn starch has also been tested in type 1 diabetic children. The comparison with a normal snack is clearly in favour of the corn starch regimen with regard to averting nocturnal hypoglycemia. It has been shown that corn starch consumption at bedtime leads to a 70% reduced number of hypoglycemic episodes at 3 AM in type 1 diabetic adults. Moreover, the regimen does not appear to compromise the glycemic control during a 4-week period, despite the fact that it is added as a supplement.

Native corn starch granules are used as the carbohydrate source. The granules range from approximately 2-32 μm in size.

Native corn starch is an odourless fine particular crystalline powder with a water content within 10-14%. The crystallinity can be observed in a polarised light microscope and particles, e.g. granules with a characteristic dark cross are evident.

The particle size together with the amount of intact granules is an important factor for the enzymatic degradation profile of native corn starch.

Starch granules are mainly made up of two components, amylose and amylopectin. Amylose has a linear structure while amylopectin is branched. Both amylose and amylopectin consist of α-(1,4)-linked glucose residues while amylopectin also has α-(1,6)-linked glucose residues. Starch granules are insoluble in cold water and swell in warm. The swelling is reversible until the temperature reaches 55-65° C.

Degradation of starch is catalysed by α-amylase. From amylose, the end products are maltose (approx. 90%) together with glucose and maltotriose. From amylopectin, the same end products are produced, together with branched oligosaccharides (α-dextrins). In man, α-amylase is present in saliva och in the small intestine. During in-vitro and in-vivo conditions the digestibility of starch depends on the source of starch as well as on the pre-treatment.

### SUMMARY OF THE INVENTION

The main object of the present invention is to provide a corn starch granulate which upon intake is capable to optimize the nocturnal glucose delivery to provide a “time” effective profylaxis for nocturnal hypoglycemia to persons with diabetes.

Another object of the invention is to provide a process for the manufacture of such corn starch granulates which will provide for optimized glucose delivery by their resistance to enzymatic degradation upon oral administration.

Yet another object of the invention is to provide a corn starch product possessing the desired degradation profile in combination with agreable taste and texture.

Still another object of the invention is to provide a process for the preparation of a corn starch product using mild operational conditions so as to maintain the primary corn starch granules intact as they appear in native corn starch.

Another object of the invention is to provide a corn starch formulation which on intake gives a desired increase in blood glucose level for a duration of up to about 6 to 8 hours.

For these and other objects which will be clear from the following disclosure the present invention provides for a process for the manufacture of a corn starch granulate possessing resistance to enzymatic degradation upon oral administration. Such process involves the following steps:

a) granulating native corn starch by cautious mixing of a granulation fluid comprising a binder, ethanol or water as a solvent, corn starch and a sweetener;

b) subjecting the granulated material resulting from step a) to wet sieving;

c) drying the granulate obtained in step b) at a temperature less than about 55° C. to avoid gelatinization of the corn starch; and

a) sizing the dried granulate from step c) by dry sieving.

In such process the binder is preferably selected from pre-geletinized corn starch, gum arabicum, guar gum, potassium alginate, carageenan, metyl cellulose, and ethyl cellulose.

Among these binders methyl cellulose and ethyl cellulose are particularly preferred, and especially ethyl cellulose.

Other features of the process according to the invention are given in the appended process claims.

The process according to the invention suitably involves a further step of pressing the granulate into tablets each weighing about 1 to 10 g.

The present invention also relates to corn starch granulate comprising primary corn starch granules as they appear in untreated native corn starch, said primary granules being agglomerated, without degradation thereof, into larger secondary granules using a binder selected from pre-gelatinized corn starch, gum arabicum, guar gum, potassium alginate, carageenan, methyl cellulose, and ethyl cellulose.

Such granulates preferably also comprise isomalt to assist in granulation and to add taste to the granulate.

It is preferred that corn starch is a major constituent in the granules and preferably constitutes more than about ⅔ by weight of said granulate.

It is also preferred to add a vegetable oil to the product to obtain a fat supplement without increasing the colesterol level, Examples of such oils are olive oil, rape seed oil, and the like, and the added amount may be up to about 3% by weight.

However, the binder need not be pre-dissolved but can be admixed together with the other components.

Other features of the corn starch product according to the present invention are found in appended claims 13 to 28.

### DETAILED DESCRIPTION OF THE INVENTION

The present invention will be described more in detail in the following by specific examples and with reference to the appended drawing. The drawing shows a diagram on the blood glucose variation as a function of time comparing an embodiment of the corn starch product of the invention and a control devoid of corn starch in accordance with the invention. These examples are not intended to limit the scope of the invention.

The Process in General

The production process for the manufacture of corn starch granulate in accordance with the invention is generally a wet granulation process. A granulation fluid consisting of binder dissolved in denaturated or non-denaturated ethanol of 70-99.5% is produced. However, the binder need not be pre-dissolved but can be admixed together with the other components. The granulation fluid as obtained is added to a powder premix consisting of native corn starch and a sweetener and blended in a mixer or other means for mixing the constituents. The moist mixture is wet sieved or gently milled in an oscillating sieve or equivalent thereto. The wet mass obtained is dried to dryness at a temperature below about 55° C. The dried mass is dry sieved or gently milled to brake up larger lumps in an oscillating sieve or similar device.

The dried granulates are then mixed with colloidal silica dioxide in an ordinary mixer, double cone mixer or similar aparatus. In a second mixing step magnesium stearate is admixed. Then tablets are compacted to give a crushing strengt of about 2-18 kp in an ordinary tablet press.

### SPECIFIC EXAMPLES

### Manufacture of Corn Starch Granulate

9.2 kg ethyl cellulose is dissolved in 28 kg ethanol (70-99.5%). 68.60 kg native corn starch and 16.25 kg isomalt are dry mixed in a mixer. After this mixing the ethanol containing ethyl cellulose is slowly added to the dry mass and mixing is continued until a uniformly wetted mass is obtained.

The wetted mass is then sized through a 1 to 2 mm screen or mill to give a wet granulate. This wet granulate is then dried on trays or in a fluidised bed at a temperature of less than about 55° C. to dryness. The dried granulate is then sized through a 1 to 2 mm screen or mill.

However, as previously indicated, the binder need not be pre-dissolved but can be admixed together with the other components.

### Preparation of Corn Starch Tablets

The dried and sieved ganulate obtained above is mixed with 1 kg colloidal silica for 10 minutes. 0.5 kg magnesium stearate is then added and mixing is carried out for about 2 minutes. The final mix obtained is tranferred to the hopper of a tablet press equipped with Ø15-25 mm punches with bevelled edges. Tablets of about 2 to 10 g are pressed to give a crushing strengt of from about 12-14 kp.

### SPECIFIC EXAMPLE 2

### Corn Starch Granulate

Corn starch granulate is manufactured as described in Example 1 above containing the following constituents given as percentage by weight.

| | |
|---|---|
| Corn search | 72.9 |
| Ethyl cellulose | 9.7 |
| Isomalt PF | 15.1 |
| Malic acid | 0.6 |
| Aroma lemon (citro) | 0.2 |
| Aspartame | 0.04 |

### Corn Starch Tablets

To a corn starch granulate having the composition given above Aerosil 200 1.0 and Mg-stearate 0.5 percent by weight are added for the transfer into tablets. The tablets have a weight of between about 2 to 10 g.

In the product described above in Example 2 corn starch has the advantage that it is an unsatisfactory carbohydrate source for the bacteria of the oral cavity thereby minimizing the risk for caries. Isomalt is added as an extra granulation component as well as sweetener. Isomalt is normally not utilized as a carbohydrate source in humans and will not significantly contribute with fast carbohydrates so as to compromise evening blood glucose levels. Furthermore, Isomalt is also a less digestable carbohydrate source for bacteria in the mouth so as to further reduce the risk for caries.

The fruit acid, such as malic acid, is used to stimulate the salivation during ingestion to reduce the perception of a “dry compound”. Furthermore, aroma is used to improve taste; lemon scent is especially well tasting in combination with the basic taste and texture of the formulation.

Aspartame was surprisingly shown to mask the “chalkyness” of the corn starch although not proving it seems as though the perception of both the “chalkyness” and the sweet sensation of aspartame occcurs in the brain at the same time thus masking the chalky taste of corn starch.

An important factor for taste and texture is the crushing strength of the tablet. A preferred range is from about 8 to about 18 kp where the texture is acceptable, whereas an optimum range of crushing strength is about 12 to about 14 kp for optimum texture.

When tested in vivo, the corn starch product of the invention results in a blood glucose profile increasing linearly from about 45 minutes to about 5 hours, whereafter it stays at the same level for at least about 2 more hours. This is totally unexpected when compared to the original release profile of native corn starch, wich has a “low hill shaped” release profile. It is also quite unexpected to observe that such small amount of corn starch as 5-20 grams will secure the blood glucose levels for such a long period of time as about 7-8 hours.

### SPECIFIC EXAMPLE 3

### Clinical Test

The patient arrives in the laboratory in the morning in fasting state and without having taken the regular morning insulin dose. For the establishment of a base line the blood glucose level will be stabilized at 5.5 to 6.5 mmol per litre with the help of a slow i.e. infusion of insulin combined with a glucose infusion. The insulin is administrated by an infusion rate, aiming at giving a blood insulin concentration of 15-20 mU/l. The glucose concentration will be locked by customary clamp technique, where blood sugar is measured every 5^{th} minute for 1 hour and the glucose infusion rate is adjusted if necessary to give the desired blood glucose concentration. Thereafter the control medication is given and the glucose clamp is continued for 6 hours.

During the test, day blood samples are withdrawn every 10^{th} minute during the first 6 hours of the experiment for glucose determination, and also every 60^{th} minute for insulin determination.

The result of the clinical test is summarized in the diagram of the appended drawing. Herein the blood glucose level in mmol/l is plotted as a function of time. 6 Tablets according to specific Ex 2 having a total weight of about 15 g and a total starch weight of about 10 g have been taken at time 0 and compared with a control not containing corn starch granulate according to the invention.

As is clear from the diagram, the blood glucose profile using the tablets of the present invention compared to the control is indeed surprisingly different and results in a pronounced increase in blood glucose level up to about 4-5 hours and then staying at the same level at least about 2 hours more. The blood glucose profile obtained by exercising the present invention indeed constitutes a great improvement in regard to the diabetes problem and greatly facilitates the treatment of diabetes with regard to the nocturnal hypoglycemia level in individuals suffering from diabetes.

Moreover, the tablets of this invention possess the unexpected feature of giving a slow release of the glucose contents of the starch in spite of the fact that the tablet is subjected to chewing.

1. A process for the manufacture of a corn starch granulate possessing resistance to enzymatic degradation upon oral administration, comprising the steps:
a) granulating native corn starch by cautious mixing of a granulation fluid comprising a binder, ethanol or water as a solvent, corn starch and a sweetener;
b) subjecting the granulated material resulting from step a) to wet sieving;
c) drying the granulate obtained in step b) at a temperature less than about 55° C. to avoid gelatinization of the corn starch; and
d) sizing the dried granulate from step c) by dry sieving.

2. A process according to claim 1, wherein said binder is selected from pre-gelatinized corn starch, gum arabicum, guar gum, potassium alginate, carageenan, methyl cellulose, and ethyl cellulose.

3. A process according to claim 1 or 2, wherein the sweetener is selected from isomalt, fructose, xylitol, and aspartame.

4. A process according to claim 2 or 3, wherein said binder is methyl cellulose or ethyl cellulose.

5. A process according to claim 4, wherein said binder is admixed into ethanol or is admixed together with the other components.

6. A process according to any one of claims 3 to 5, wherein said sweetener is isomalt and, optionally, aspartame.

7. A process according to claim 1, comprising the steps:
a) granulating native corn starch by cautious mixing of a granulating fluid comprising ethyl cellulose, preferably dissolved in ethanol and a pre-mix of corn starch and isomalt as a sweetener;
b) subjecting the granulated material resulting from step a) to wet sieving by sizing through a screen or mill within the range about 1 to 3 mm;
c) drying wet granulate obtained from step b) at a temperature of less than about 55° C.; and
d) sizing the dried granulate from step c) on a screen or mill within the range about 1 to 2 mm.

8. A process according to any preceding claim, comprising the further step of pressing the granulate into tablets each weighing about 1 to 10 g.

9. A process according to claim 8, wherein said further step is comprised by mixing the granulate with colloidal silica for improving flow and with magnesium stearate as a lubricant before pressing the granulate into tablets.

10. A process according to claim 8 or 9, wherein the tablets are pressed to give a crushing strength of about 2 to 18 kp.

11. Corn starch granulate whenever obtained by the process of any one of claims 1 to 7.

12. Corn starch granulate tablets whenever obtained by the process of any one of claims 8 to 10.

13. Corn starch granulate comprising primary corn starch granules as they appear in untreated native corn starch, said primary granules being agglomerated, without degradation thereof, into larger secondary granules to form a granulate using a binder selected from pre-gelatinized corn starch, gum arabicum, guar gum, potassium alginate, carageenan, methyl cellulose, and ethyl cellulose.

14. Corn starch granulate according to claim 13, further comprising isomalt to assist in granulation and to add taste to the granules.

15. Corn starch granulate according to claim 13 or 14, wherein corn starch is a major constituent, and where in the binder is present in an amount of about 5 to 15% by weight based on the weight of the granulate.

16. Corn starch granulate according to claim 15, wherein corn starch constitutes more than about half and preferably more than about ⅔ by weight of said granulate.

17. Corn starch granulate according to any one of claims 14 to 16, wherein isomalt is present in an amount of about 5 to 30% by weight based on the weight of the granulate.

18. Corn starch granulate according to any one of claims 12 to 17, further comprising aspartam to further add to the taste of the granulate.

19. Corn starch granulate according to claim 18, wherein aspartam is present in an amount of about 0.01 to 0.1% by weight based on the weight of the granulate.

20. Corn starch granulate according to any one of claims 13 to 19, further comprising a fruit acid to add flavour to the granulate.

21. Corn starch granulate according to claim 20, wherein said fruit acid is selected from malic, tartaric, and citric acids.

22. Corn starch granulate according to claim 20 or 21, wherein said acid is present in an amount of about 0.1 to 1.5% by weight based on the weight of the granulate.

23. Corn starch granulate according to any one of claims 13 to 23, further comprising an aroma substance.

24. Corn starch granulate according to claim 23, wherein said substance is of citrus origin.

25. Corn starch granulate according to claim 23 or 24, wherein said substance is present in an amount of about 0.1 to 1.0% by weight based on the weight of the granulate.

26. Corn starch granulate according to any one of claims 13 to 25, wherein a majority of the primary granules have an average cross dimension of from about 15 μm to 25 μm, and wherein a majority of the secondary granulate have an average cross dimension of from about 0.3 to 1 mm.

27. Corn starch granulate according to any one of claims 13 to 26, wherein the binder is ethyl cellulose.

28. Corn starch granulate according to any one of claims 13 to 27, presented in the form of tablets, each having a weight of from about 1 g to 10 g.
